(19) United States
(12) Patent Application Publication
(10) Pub. No.: US 2023/0302187 A1
(43) Pub. Date: Sep. 28, 2023

(54) UV DISINFECTION DEVICE

(71) Applicant: Stuart Victor SQUIRES, Bergen (NO)

(72) Inventor: Stuart Victor SQUIRES, Bergen (NO)

(21) Appl. No.: 18/023,320

(22) PCT Filed: Aug. 26, 2021
(86) PCT No.: PCT/CA2021/051188

§ 371 Date: Feb. 24, 2023

Related U.S. Application Data
(60) Provisional application No. 63/071,015, filed on Aug. 27, 2020.

(51) Int. Cl. A61L 9/20; A61M 16/06

(52) U.S. Cl. CPC A61L 9/20; A61M 16/06; A61L 2209/111; A61L 2202/122

(57) ABSTRACT

An ultraviolet (UV) disinfection device comprises a housing shaped to define a chamber. The chamber comprises an inlet, an outlet, and one or more dividers arranged to define one or more flow paths between the inlet and the outlet. The dividers are preferably made of a UV transparent material. A UV radiation emitter is provided inside the chamber of the UV disinfection device to deliver UV radiation to air flowing through the chamber.

### REFERENCE TO RELATED APPLICATIONS

This application claims priority from U.S. Application No. 63/071,015 filed on 27 Aug. 2020 and entitled UV DISINFECTION DEVICE. For purposes of the United States, this application claims the benefit under 35 U.S.C. § 119 of U.S. Application No. 63/071,015 filed on 27 Aug. 2020 and entitled UV DISINFECTION DEVICE which is hereby incorporated herein by reference for all purposes.

### TECHNICAL FIELD

The present invention relates generally to ultraviolet (UV) disinfection devices, and more particularly, to a UV chamber having UV radiation emitters such as UV light emitting diodes (UV-LEDs). Some embodiments have example applications for disinfecting airborne pathogens. Some embodiments may be embodied as part of respiratory personal protective equipment (PPE).

### BACKGROUND

UV radiation is known to be effective in sanitizing germs such as bacteria, fungi and viruses by damaging the RNA/DNA of the germs such that they become incapable of reproducing. Accordingly, it is common to use UV radiation for irradiating fluids in a UV disinfection device (e.g. for applications such as water disinfection).

While it is known to use UV radiation for sanitization applications, state of the art UV disinfection devices share several common problems. One problem with some UV disinfection devices is that they are not energy efficient. Another problem with some UV disinfection devices is that they are not environmentally friendly (e.g. some UV disinfection devices use hazardous materials like mercury). Another problem with some UV disinfection devices is that they are expensive to manufacture. Another problem with some UV disinfection devices is that they are not portable. Another problem with some UV disinfection devices is that they are not capable of being embodied as part of a larger system. Another problem with some UV disinfection devices is that they do not deliver sufficient amounts of UV radiation to sanitize germs. Another problem is that some UV disinfection devices that emit UV radiation (e.g. mercury lamps) take several minutes to “warm up” before they are able to reach optimal disinfection rates.

There remains a need for UV disinfection devices that are energy efficient, effective for sanitizing germs, environmentally friendly, portable and/or capable of being embodied as a part of a larger system. There also remains a need for cost-effective UV disinfection devices which possess such properties.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Aspects of the invention include, without limitation, disinfection devices operated by UV radiation emitters (e.g. UV light emitting diodes), and personal protective equipment (PPE) comprising UV disinfection devices.

One aspect of the invention relates to an ultraviolet (UV) disinfection device. The UV disinfection device comprises a housing shaped to define a chamber extending in a longitudinal direction, an inlet located at a first longitudinal end of the chamber, an outlet located at a second longitudinal end of the chamber, a radiation emitter, and one or more dividers. The radiation emitter comprises one or more UV radiation sources. The radiation emitter is located relatively distally from the first longitudinal end of the chamber and relatively proximately to the second longitudinal end of the chamber. The radiation emitter emits UV radiation optically oriented toward the first longitudinal end of the chamber. The dividers are made of a UV transparent material and spaced apart in a transverse direction to define flow paths between the inlet and the outlet.

In some embodiments, the dividers include first and second dividers extending from the radiation emitter toward the first longitudinal end. The radiation emitter may comprise first and second channels located at opposing edges of the radiation emitter that allow the first and second channels to be retained therein. In some embodiments, the dividers include third and fourth dividers extending from an interior surface of the housing at the first longitudinal end of the chamber toward the second longitudinal end. In some embodiments, the dividers are spaced apart in the transverse direction to define two or more flow paths between the inlet and the outlet. Each of the two flow paths may comprise respective serpentine shaped segments. The serpentine shaped segments of each of the two flow paths may be shaped to meander in opposing directions. In some embodiments, the two flow paths contain segments which overlap with each other. The overlapping segments of the first and second flow paths may be located at the inlet.

In some embodiments, the inlet and the outlet comprise respective axes which are generally parallel to the longitudinal direction. The axes of the inlet and the outlet may be parallel with each other. The axes of the inlet and the outlet may be aligned. In some embodiments, the radiation emitter comprises a principal optical axis which is parallel to the axes of the inlet and the outlet.

In some embodiments, the UV radiation source comprises UV light emitting diodes (UV-LEDs). The UV-LEDs may be arranged in a rectangular array. In some embodiments, the housing comprises internal surfaces which are made of a UV reflective material. In other embodiments, the housing comprises internal surfaces adapted to receive inserts made of a UV reflective material. The UV reflective material may be aluminum, silver, polytetrafluoroethylene (PTFE), and/or aluminum coated mylar.

In some embodiments, the UV disinfection device comprises a UV blocker outside of the inlet. The UV barrier is at least partially made of a UV absorbing material. In some embodiments, the UV barrier comprises a UV reflective material located between the UV absorbing material. In some embodiments, the UV reflective material is aligned with the axis of the inlet. In some embodiments, the UV disinfection device comprises one or more UV blockers made of glass beads pressed together in a packed configuration. The UV blocker may be located at the inlet and/or the outlet.

Another aspect of the invention relates to a personal protective equipment comprising a face mask, a disinfection chamber, a substrate supporting one or more UV radiation sources, and a plurality of dividers spaced apart in the chamber. The divider has a first port that places the disinfection chamber in fluid communication with the face mask, and a second port that places the disinfection chamber in fluid communication with ambient environment. The substrate is located in the chamber. The dividers are made of a UV transparent material. The dividers and the substrate are arranged in the chamber to collectively define multiple flow paths between the first port and the second port.

In some embodiments, the personal protective equipment comprises a fan configured to move exhaled air from the face mask through the disinfection chamber to the ambient environment. A one-way valve may be provided between the disinfection chamber and the face mask to prevent air from back flowing from the disinfection chamber toward the face mask.

In some embodiments, the personal protective equipment comprises a fan configured to move air in the ambient environment through the disinfection chamber and toward the face mask. A one-way valve provided between the disinfection chamber and the face mask to prevent the air from back flowing from the face mask toward the disinfection chamber.

In some embodiments, the personal protective equipment comprises a materials filter located at the first port and/or the second port. In some embodiments, the personal protective equipment comprises an electrostatics filter located at the first port and/or the second port.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic top view of an ultraviolet (UV) disinfection device according to an example embodiment. FIG. 1A shows a perspective cutaway view of an example implementation of the UV disinfection device shown in FIG. 1. FIG. 1B shows a perspective view of an example embodiment of the FIG. 1A implementation of the UV disinfection device.

FIG. 2 is a plan view of an exemplary UV emitter which forms part of the FIG. 1 UV disinfection device.

FIG. 3 is a block diagram depicting various components of a UV disinfection device according to an example embodiment.

FIG. 4 illustrates an exemplary UV blocker which may be provided as part of the FIG. 1 UV disinfection device. FIG. 4A illustrates an alternative embodiment of a UV blocker. FIG. 4B is a plan view of the UV blocker shown in FIG. 4B.

FIG. 5A is a schematic diagram depicting a personal protection equipment (PPE) comprising the UV disinfection device shown in FIG. 1 and configured for disinfecting air to be inhaled. FIG. 5B is a schematic diagram depicting a personal protection equipment (PPE) comprising the UV disinfection device shown in FIG. 1 and configured for disinfecting exhaled air.

FIGS. 6A-E show the results for various simulation experiments conducted by the inventor.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention relates to UV disinfection devices which use UV radiation emitters such as UV light emitting diodes (LEDs) as the source of UV radiation. Light emitting diodes (LEDs) are solid state radiation sources which release photons when an electric potential is applied across the LED. LEDs may be designed or otherwise operated to emit radiation in the UV region of the electromagnetic spectrum. Advantageously, UV-LEDs are small, energy efficient, inexpensive to manufacture, and more environmentally friendly than traditional UV lamps which typically contain mercury. UV-LEDs also do not have a “warm up” time. While UV-LEDs may be used in some embodiments, UV disinfection devices described herein may comprise other types of UV radiation emitters. The other type of UV radiation emitters may include, for example, UV lamps, UV lasers, tunable vacuum UV (VUV), and plasma UV.

Another aspect of the invention relates to respiratory personal protective equipment (PPE) which include a UV disinfection device of the type described herein. PPE are useful for preventing germs (e.g. bacteria, fungi, viruses, etc.) from entering the human body through the respiratory tract and/or preventing germs from spreading through the air, but the supply of PPE can become limited in situations like a pandemic. When supply of PPE is limited, users may be forced to use sub-standard masks, thereby increasing the risk of infection. Advantageously, PPE which include a UV disinfection device are reusable and do not obstruct the user's ability to breath or speak (e.g. due to the lack of need to include a materials filter). Advantageously, such PPE neutralizes pathogens as opposed to capturing pathogens with a materials filter which needs frequent replacement, can pose a bio-hazard after use and can be difficult to source in situations like a pandemic. Advantageously, such PPE can be worn continuously for extended periods of time (e.g. on a bus during a user's commute to work, during an airplane flight, during work, etc.) without causing discomfort to the user.

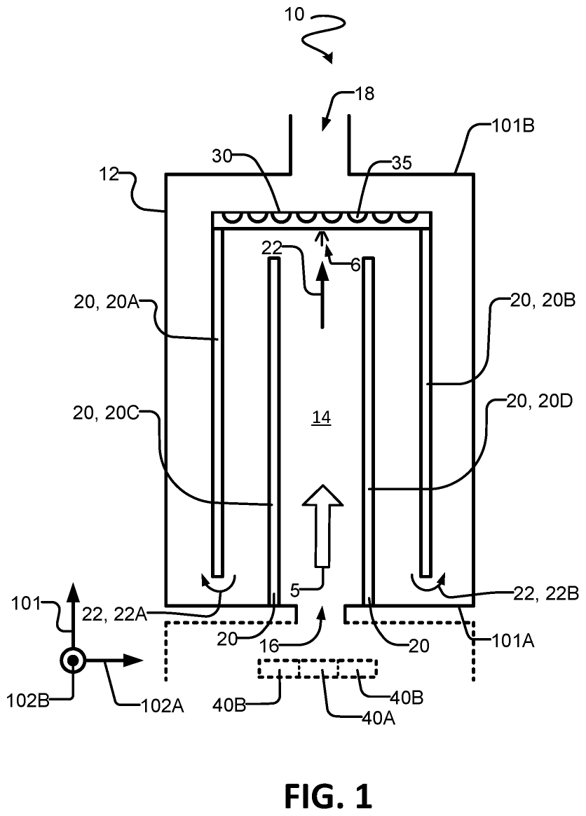

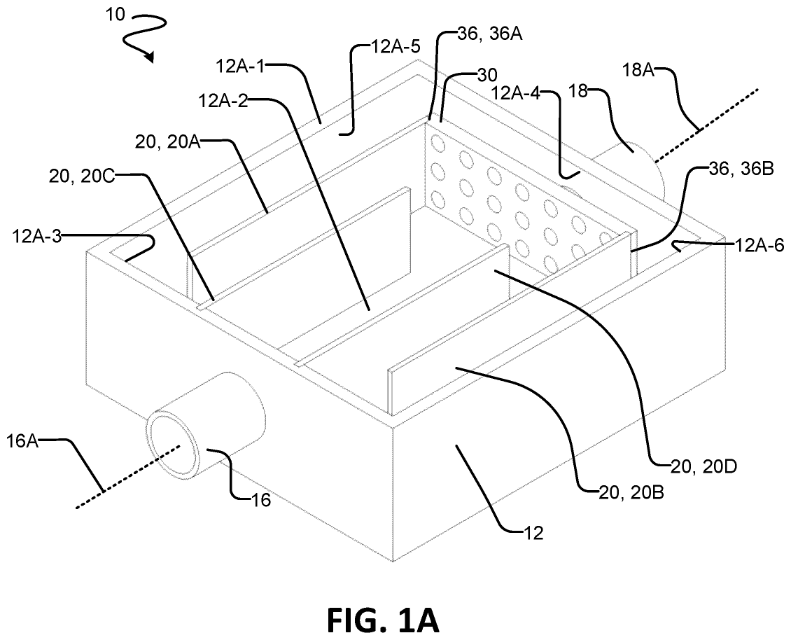

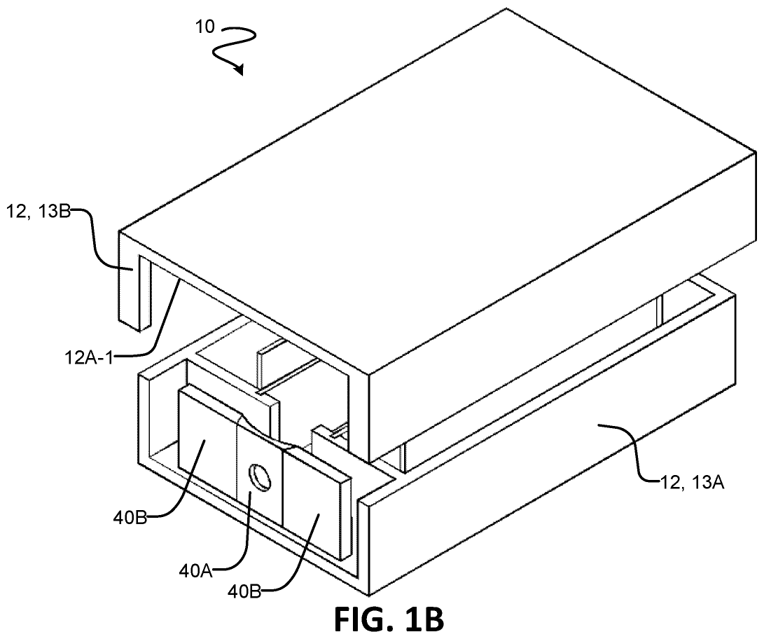

FIG. 1 is a schematic top view of an ultraviolet (UV) disinfection device 10 according to an example embodiment of the invention. FIG. 1A shows a perspective view of an example implementation of the UV disinfection device 10 shown in FIG. 1. UV disinfection device 10 has example applications for neutralizing unwanted particles (e.g. pathogens, bacteria, viruses, chemical contaminants, etc.) from fluid (e.g. air) 5 flowing through UV disinfection device 10. Device 10 is typically embodied as a part of a larger system. For example, device 10 may be embodied as a part of respiratory personal protective equipment (PPE) (e.g. a face mask, a hazmat suit, etc.), a home air purification system, a portable air purifier, etc.

As depicted in FIG. 1, UV disinfection device 10 comprises a housing 12 which defines a chamber 14 extending generally in a longitudinal direction 101. Chamber 14 extends between a first port 16 and a second port 18 of chamber 14. In the example embodiment illustrated in FIG. 1, first port 16 acts as an inlet to housing 12 and second port 18 acts as an outlet for housing 12. For the purposes of facilitating the description, first port 16 may be referred to herein as “inlet” and second port 18 may be referred to herein as “outlet”. However, it should be understood that the functions of first port 16 and second port 18 are interchangeable within the scope of the present invention. That is housing 12 may be designed or otherwise configured to either allow air 5 to enter chamber 14 through first port 16, flow through chamber 14, and exit chamber 14 at second port 18, or allow air 5 to enter chamber 14 through second port 18, flow through chamber 14, and exit chamber 14 at first port 16.

UV disinfection device 10 comprises one or more dividers 20 located in chamber 14 of housing 12. In a currently preferred embodiment, UV disinfection device 10 comprises a plurality of dividers 20 that are spaced apart from each other within chamber 14. Dividers 20 are spaced in chamber 14 to define one or more flow paths 22 for guiding the direction(s) of fluid flow between inlet 16 and outlet 18. Dividers 20 are typically made of a UV transparent material as described in more detail elsewhere herein. Flow paths 22 may be shaped by the arrangement of dividers 20. As depicted in FIG. 1, the various segments of flow paths 22 may be defined by one of more of: dividers 20, interior surface 12A, and emitter 30. Dividers may include those which extend (or have parts that extend) along longitudinal direction 101 and/or those which extend (or have parts that extend) along transverse directions 102A, 102B (i.e. directions which are generally perpendicular to longitudinal direction 101) as described in more detail elsewhere herein. A UV emitter 30 is located in chamber 14 (i.e. inside housing 12) to deliver UV radiation 6 to air 5 as air 5 flows through flow paths 22 in chamber 14.

In the example embodiment illustrated in FIG. 1, UV disinfection device 10 comprises first and second dividers 20A, 20B extending from UV emitter 30 and third and forth dividers 20C, 20D extending from a first longitudinal end 101A of chamber 14. First and second dividers 20A, 20B may be coupled to a top surface 12A-1 of chamber 14, a bottom surface 12A-2 of chamber 14 and/or UV emitter 30. Third and fourth dividers 20C, 20D may be coupled to a top surface 12A-1 of chamber 14, a bottom surface 12A-2 of chamber 14 and/or a front surface 12A-3 of chamber 14 (i.e. the surface defining first longitudinal end 101A).

In the example embodiment illustrated in FIG. 1, each of dividers 20A, 20B, 20C, 20D extend in longitudinal direction 101. Dividers 20A, 20B, 20C, 20D may extend in parallel relative to one another. Dividers 20A, 20B, 20C, 20D may be spaced apart along transverse direction 102A to define two flow paths 22A, 22B between inlet 16 and outlet 18 as depicted in FIG. 1.

Housing 12 may be designed to provide a chamber 14 of any suitable shape, size, and/or dimension. Although not necessary, housing 12 is typically designed to provide a cuboid shaped chamber 14 as depicted in FIG. 1A. In such designs, chamber 14 may be characterized by a length (e.g. a longest side) extending along longitudinal direction 101, a width (e.g. a shorter side) extending along a first transverse direction 102A, and a thickness (e.g. a shortest side) extending along a second transverse direction 102B (i.e. a direction generally orthogonal to first transverse direction 102A and longitudinal direction 101). In some embodiments, chamber 14 has a length which is in the range of about 15 cm to 20 cm, a width which is in the range of about 5 cm to 10 cm, and a thickness which is in the range of about 2 cm to 10 cm. Such dimensions may be suitable in embodiments where device 10 is provided to form part of a PPE.

In some embodiments, one or more interior surfaces 12A of housing 12 (which collectively define the volume of chamber 14) is coated with or otherwise comprises a suitable reflective material that is reflective to radiation emitted by UV emitter 30. For example, housing 12 may comprise six interior surfaces 12A-1, 12A-2, 12A-3, 12A-4, 12A-5, 12A-6 as depicted in the example embodiment shown in FIG. 1A, and one or more of the six interior surfaces 12A-1, 12A-2, 12A-3, 12A-4, 12A-5, 12A-6 of housing 12 may comprise a UV reflective material. Examples of suitable reflective materials include, but are not limited to aluminum, silver, polytetrafluoroethylene (PTFE), aluminum coated mylar, etc.

In some embodiments, the one or more interior surfaces 12A of housing 12 comprises UV reflective inserts (e.g. thin and lightweight pieces of UV reflective material such as aluminum) which are removably coupled to housing 12. That is, the reflective inserts may be coupled (e.g. attached by an adhesive, mechanically fastened, etc.) to the interior surfaces 12A of housing 12. Conveniently such inserts may be swapped out with new inserts if they become less reflective over time (e.g. due to oxidation). In embodiments where housing 12 comprises UV reflective inserts, housing 12 may be made of a non-UV reflective material to reduce costs of manufacture.

In the example illustrated in FIG. 1A, chamber 14 comprises an inlet 16 which allows air 5 to enter chamber 14 and an outlet 18 which allows air 5 to exit chamber 14. Although not necessary, inlet 16 and outlet 18 are typically provided at opposing ends of chamber 14. For example, inlet 16 may be provided at a first longitudinal end 101A of chamber 14 and outlet 18 may be provided at a second longitudinal end 101B of chamber as shown in FIG. 1. In some embodiments, the distance between inlet 16 and outlet 18 spans the entire length of chamber 14.

In some embodiments, the axis 16A of inlet 16 is parallel to longitudinal direction 101 (i.e. the axis 16A of inlet 16 is oriented to point in the direction of the length of chamber 14). In some embodiments, the axis 18A of outlet 18 is parallel to longitudinal direction 101 (i.e. the axis 18A of outlet 18 is oriented to point in the direction of the length of chamber 14). In some embodiments, the axis 16A of inlet 16 and the axis 18A of outlet 18 are parallel to each other. In some embodiments, the axis 16A of inlet 16 and the axis 18A of outlet 18 are aligned as depicted in FIG. 1A.

Preferably the location and/or orientation of inlet 16 and outlet 18 are provided or otherwise configured based on the location/orientation of dividers 20 (which define the geometry or shape of flow paths 22) and/or the location/orientation of UV emitter 30 to allow UV emitter 30 to deliver a suitable amount of UV radiation 6 to air 5 as air 5 flows through flow paths 22 in chamber 14.

As described above, chamber 14 comprises one or more dividers 20 arranged to define one or more flow paths 22 between inlet 16 and outlet 18. Unless context dictates otherwise, a flow path 22 described herein refers to a continuous path between inlet 16 and outlet 18 of chamber 14, allowing air 5 to flow therebetween and through chamber 14. Each flow paths 22 may comprise segments which extend generally along longitudinal direction 101, segments which extend generally along a first transverse direction 102A and/or segments which extend generally along a second transverse direction 102B. That is, dividers 20 may be arranged to define flow paths 22 which are serpentine shaped or flow paths 22 which have segments that are serpentine shaped. Such serpentine shaped flow paths 22 may comprise rectangular corners as shown in FIG. 1, or other crenulated shapes such as zig zags, sinusoidal shapes, etc.

Arranging dividers 20 to provide flow paths 22 which are shaped in such manner can advantageously increase the overall path length between inlet 16 and outlet 18 (i.e. increase the distance air 5 is required to travel to reach outlet 18 from inlet 16). Increasing the overall path length (of flow paths 22) between inlet 16 and outlet 18 can advantageously increase the amount of time required for air 5 to flow through housing 12 to thereby increase the dose of UV radiation 6 delivered to air 5 as air 5 flows through housing 12. Arranging dividers 20 to provide flow paths 20 shaped in manners described herein can help ensure that air 5 receives sufficient irradiation as it flows through chamber 14 (e.g. by preventing air 5 from taking “short cuts” as it flows between inlet 16 and outlet 18). Flow paths 20 may also advantageously help create guaranteed airflow pathlines which can provide more laminar flow and less turbulence.

In some embodiments, the one or more dividers 20 are arranged to define flow paths 22 which comprise segments that overlap with each other. For example, the plurality of dividers 20 may be arranged to define two flow paths 22A, 22B which have initial segments (e.g segments proximate to inlet 16) that overlap as shown in FIG. 1. In some embodiments, first flow path 22A and second flow path 22B respectively comprise segments which extend generally along longitudinal direction 101 and segments which extend generally along first transverse direction 102A. In some embodiments, first flow path 22A and second flow path 22B comprise respective serpentine segments shaped to meander in opposing directions along an axis such as transverse axis 102A. In some embodiments, first flow path 22A and second flow path 22B are symmetrical about an axis of symmetry. For example, first flow path 22A and second flow path 22B may be symmetrical about a common axes 16A, 18A of inlet 16 and outlet 18 (i.e. in embodiments where inlet 16 and outlet 18 are aligned) as depicted in FIG. 1A.

Preferably chamber 14 comprises dividers 20 which are made of suitable UV-transparent materials such as fused silica or quartz. UV-transparent dividers 20 can advantageously allow UV emitter 30 to deliver greater amounts of UV radiation 6 to air 5 (compared to providing non-UV-transparent dividers) as air 5 flows along flow paths 22. Dividers 20 are typically planar-shaped as shown in FIG. 1A, but this is not necessary. Dividers 20 may have any suitable shape (e.g. divider 20 may be concave shaped, convex shaped, etc.). Planar-shaped dividers 20 may be preferred in some cases to provide advantages such as ease of manufacturing and/or ease of assembly.

In some embodiments, chamber 14 comprises reflective interior surfaces 12A and UV-transparent dividers 20. This combination allows UV radiation 6 emitted by UV emitter to be reflected off of the interior surfaces 12A in a variety of different directions. In some cases, small dust particles can shade an unwanted particle (e.g. pathogen) from exposure to radiation impinging from a certain direction, so it is desirable to direct radiation toward the unwanted particle from multiple directions. Providing UV-transparent dividers 20 and reflective interior surfaces 12A in combination allows UV radiation 6 emitted by UV emitter to impinge on an unwanted particle from multiple directions, thereby increasing the effectiveness of device 10.

As described above, a UV emitter 30 is located within chamber 14 and oriented to direct UV radiation 6 toward air 5 as air 5 flows through chamber 14. UV emitter 30 emits UV radiation 6 that may be optically oriented in directions that are generally parallel or antiparallel (i.e. parallel but in opposite direction) to the primary direction of air flow (i.e. along longitudinal direction 101). For example, UV emitter 30 may emit UV radiation 6 that is optically oriented toward inlet 16 as shown in FIG. 1. The optical orientation of UV radiation 6 may optionally incorporate the use of optical elements such as lenses, reflectors, and waveguides located in the optical path between a UV radiation source 35 and an output of UV emitter 30. In some embodiments, UV radiation 6 that is optically oriented in a particular direction has a maximal intensity in that particular direction.

Alternatively, UV emitter 30 may emit UV radiation 6 that is optically oriented in a direction which is generally perpendicular to the primary direction of air flow within chamber 14.

UV emitter 30 is typically located at or proximate to a first longitudinal end of chamber 14 and oriented to direct UV radiation 6 toward an opposing second longitudinal end of chamber 14. For example, UV emitter 30 may be located between inlet 16 and outlet 18, but relatively proximate to the second longitudinal end 101B (i.e. at the longitudinal end of chamber 14 that is relatively proximate to outlet 18 and/or relatively distal from inlet 16 when compared to the opposing longitudinal end 101A of chamber 14) as shown in FIG. 1A. Such configuration allows UV emitter 30 to deliver sufficient amounts of UV radiation 6 to air flowing through flow paths 32. Such configuration, in combination with UV transparent dividers 20, creates geometrically advantageous flow paths 22 for air 5 to receive sufficient exposure to UV radiation 6.

In some embodiments, UV emitter 30 is removably coupled to housing 12. That is, UV emitter 30 may be inserted into and removed from chamber 14. In such embodiments, housing 12 includes a mean for accessing chamber 14.

FIG. 1B depicts an example embodiment of a UV disinfection device 10 having a means for accessing chamber 14. In the example embodiment illustrated in FIG. 1B, device comprises a cover 13A that is removably mounted on a base 13B to form housing 12. This allows a user to access chamber 14 by separating cover 13A from base 13B. Cover 13A may be secured to base 13B by way of a mechanical coupling such as a snap-fit, a bolted connection, etc. When cover 13A is mounted on base 13B, cover 13A may provide the upper surface 12A-1 of housing 12 as depicted in FIG. 1B.

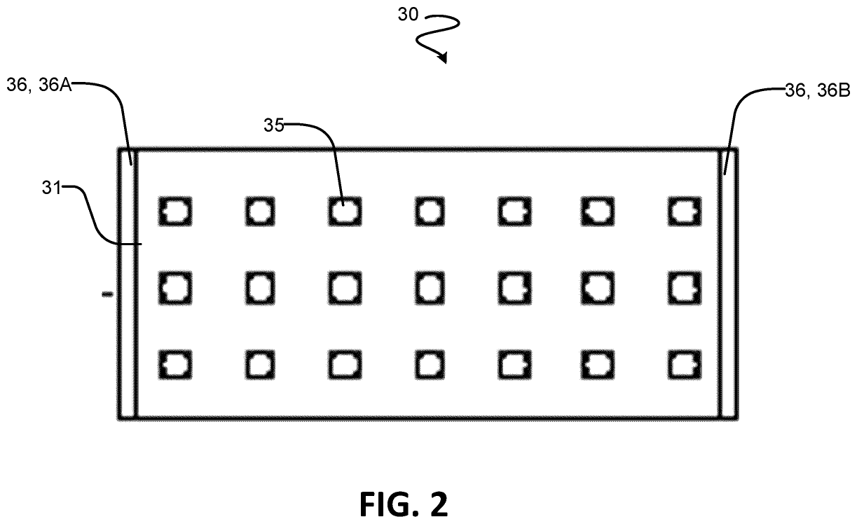

FIG. 2 is a plan view of a UV emitter 30 according to an example embodiment of the invention. In the example embodiment illustrated in FIG. 2, UV emitter 30 comprises a plurality of UV light emitting diodes (UV-LEDs) 35 mounted on a substrate 31. In general, UV emitter 30 may comprise one or more UV radiation emitting sources. UV-LEDs 35 may be configured or otherwise designed to emit radiation 6 having any wavelength in the UV spectrum. For example, the UV-LEDs 35 may emit radiation 6 in the UV-C range (e.g. radiation having wavelengths on the order of about 100 nm to about 280 nm) for bacterial or viral disinfection applications.

As shown in FIG. 2, UV-LEDs 35 may be arranged in an array on substrate 31. For example, UV-LEDs 35 may be arranged in an m×n array, where m is the number of rows in the array and n is the number of columns in the array. In some embodiments, m is a number ranging from 1 to 5 (e.g. 2, 3, 4) and n is a number ranging from 1 to 10 (e.g. 2, 3, 4, 5, 6, 7, 8, 9). Arranging UV-LEDs 35 in such manner allows UV emitter 30 to direct UV radiation 6 toward multiple different segments of flow paths 22. That is, different UV-LEDs of UV emitter 30 may direct UV radiation 6 that is optically oriented toward different segments of flow paths 22. As depicted in FIG. 1, for example, some UV-LEDs 35 may direct UV radiation 6 that is optically oriented toward a segment of a first flow path 22A defined by first divider 20A and third divider 20C, while other UV-LEDs 35 may direct UV radiation 6 that is optically oriented toward a segment of a second flow path 22B defined by second divider 20B and fourth divider 20D.

In the example embodiment shown in FIG. 2, UV-LEDs 35 are arranged in a 3×7 array. UV-LEDs 35 may be provided in any suitable arrangement on substrate 31 to deliver a suitable amount of UV dose to air 5 flowing through device 10.

Providing a UV emitter 30 having multiple spaced apart UV-LEDs 35 (e.g. UV-LEDs 35 that are spaced apart along transverse direction 102A) in combination with UV-transparent dividers 20 can, in some cases, provide device 10 with some advantages. For example, UV radiation 6 emitted by each UV-LED 35 can advantageously pass through dividers 20 into different segments of a flow path 22 and/or different flow paths 22A, 22B. This allows air 5 flowing through different flow paths 22 to receive UV radiation 6 from different UV-LEDs 35. With this design, failure of a single UV-LED 35 will not adversely affect the overall effectiveness of device 10 since adjacent UV-LEDs 35 will be able to deliver sufficient amounts of UV radiation 6 to air flowing through flow paths 22 in place of the failed UV-LED 35. If dividers 20 were not made of a UV-transparent material, then failure of a single UV-LED 35 would cause the segment of flow path 22 directly in front of the failed UV-LED 35 to receive insufficient amounts of UV radiation 6. In such situations, a pathogens traveling along the flow path 22 may not receive sufficient irradiation exposure.

In some embodiments, UV-LEDs 35 are arranged on substrate 31 in a manner that allows heat to be channeled away from UV-LEDs 35 and towards a heat sink 33 as described in more detail elsewhere herein.

In some embodiments, UV-LEDs 35 are (individually) removably mounted on substrate 31 (i.e. UV-LEDs 35 may be dismounted from substrate 31). In such embodiments, certain UV-LEDs 35 may be replaced or substituted in and out of UV emitter (e.g. by separating cover 13A from base 13B to remove UV emitter 30 from chamber 14) to allow UV emitter 30 to emit desirable amounts of UV radiation 6, desirable intensities of UV radiation 6 and/or desirable wavelengths of UV radiation 6.

In some embodiments, UV emitter 30 comprises one or more channels 36 designed to retain an end portion of a divider 20 therein. Channels 36 may be formed on substrate 31. Channels 36 may be formed near the edges of UV emitter 30 so that UV-LEDs 35 are located between channels 36. In the example embodiment illustrated in FIG. 2, UV emitter comprises a first channel 36A extending along on a first edge of UV emitter 30 and a second channel 36B extending along an opposing second edge of UV emitter 30. First channel 36A is shaped to retain a longitudinal end of first divider 20A (e.g. see FIG. 1A). Second channel 36B is shaped to retain a longitudinal end of second divider 20B (e.g. see FIG. 1A). In such embodiments, the first and second dividers 20A, 20B and third and forth dividers 20C, 20D (extending from an inner surface 12A of housing 12 at first longitudinal end 101A) may be spaced within chamber 14 to collectively define flow paths 22.

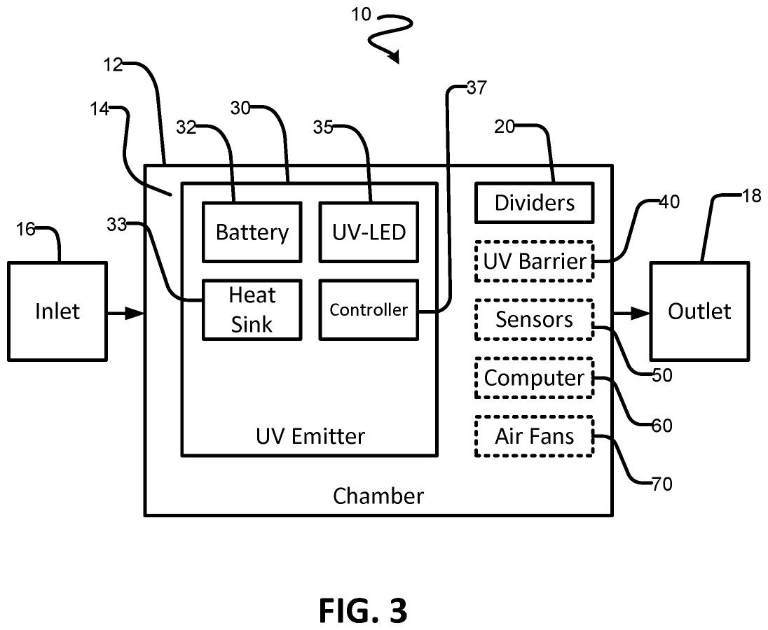

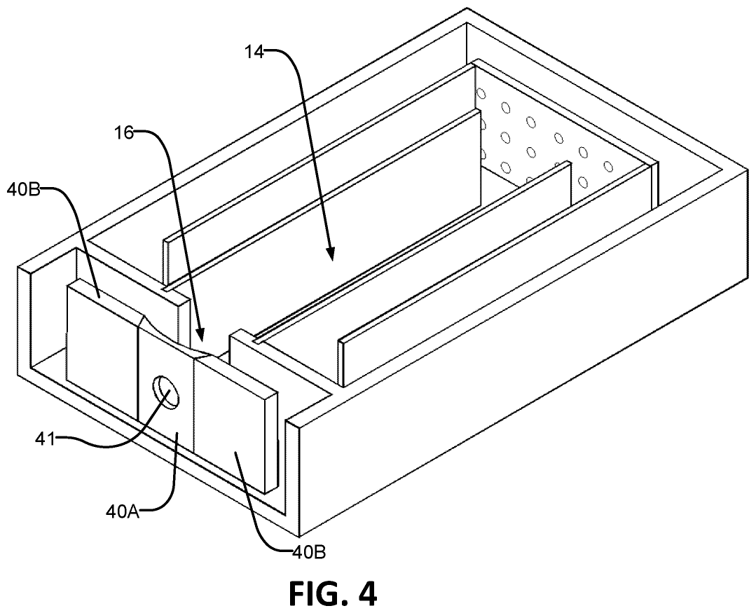

Referring now to FIG. 3, UV-LEDs 35 are typically operated by a power source 32 (e.g. a battery) that forms a part of UV emitter 30. In some embodiments, power source 32 is a rechargeable DC power source. In some embodiments, power source is 32 removably coupled to UV-LEDs 35 and/or UV emitter 30. In such embodiments, power source 32 may be replaced with a new one if power source 32 becomes worn out. Power source 32 may comprise mechanisms which help facilitate continuous operation of UV emitter even when power source 32 becomes worn out. For example, power source 32 may comprise a main battery and a temporary charge storage device (e.g. a supercapacitor, an ultracapacitor, etc.) which temporarily supplies power to UV-LEDs 35 when the main battery needs to be replaced. As another example, power source 32 may comprise two or more batteries in some embodiments. In such embodiments, the individual batteries of power source 32 may be replaced during non-overlapping times without turning off UV-LEDs 35.

Power source 32 may alternatively be an AC power source (i.e. UV-LEDs 35 and may be operated by plugging device 10 straight into an AC wall plug) or an external power bank (e.g. an external battery) which is plugged into device 10 to operate UV-LEDs 35.

In some embodiments, power source 32 is automatically disconnected from UV emitter 30 upon separation of cover 13A from base 13B. This can help ensure safe operation of device 10.

Preferably UV emitter 30 comprises one or more heat sinks 33 in thermal contact with substrate 31. Heat sink 33 is provided to improve the performance of UV-LEDs 35. Heat sink 33 may improve the performance of UV-LEDs 35 (and UV emitter 30 and device 10) by dissipating heat away from the UV-LEDs 35 to maintain the UV-LEDs 35 at a desirable temperature. Since high ambient temperatures can reduce the light output of UV-LEDs 35, it is generally desirable to operate UV-LEDs at relatively low temperatures (e.g. below about 30° C.).

In some embodiments, housing 12 acts as or otherwise provides some of the functions of heat sink 33. For example, housing 12 may comprise surfaces 12A which are made of a suitable thermally conductive material (e.g. aluminum) and in contact with UV emitter 30. For example, UV emitter 30 may be located between thermally conductive surfaces 12A as shown in FIG. 1A.

In some embodiments, UV emitter 30 comprises a casing which houses UV-LEDs 35 and substrate 31. In these embodiments, the casing is at least partially made of a suitable UV-transparent material (e.g. fused silica or quartz) to provide an optical window for UV radiation 6 emitted by UV-LEDs 35 to pass therethrough.

UV emitter 30 may also optionally comprise an electronic controller 37. Controller 37 may be formed on substrate 31. Controller 37 is operable to control the one or more UV-LEDs 35. For example, controller 37 may be operated to turn the UV-LEDs 35 off when device 10 is turned OFF (e.g. via a power button located on housing 12) and/or when cover 13A is separated from base 13B (e.g. to replace UV emitter 30, UV-LEDs 35, dividers 20, etc.). Controller 37 may be configured to receive an external control signal (e.g. a wireless control signal) from sensors 50 and/or an external controller such as software installed on a computer, a mobile application installed on a smartphone, etc. Controller 37 may be operated to control the one or more UV-LEDs 35 based on the external control signal. Controller 37 may be operated to control the UV-LEDs 35 independently from one another to help avoid issues such as thermal runaway (e.g. a problematic UV-LED 35 can be turned off individually without having to reduce the power output of UV emitter 30 and without turning off the other UV-LEDs 35).

In some embodiments, controller 37 is operable to control the number of UV-LEDs 35 which are turned ON or OFF at the same time to reduce the amount of energy consumed by UV emitter 30. For example, controller 37 may be operated to turn OFF certain UV-LEDs 35 when device 10 is located at an environment where less unwanted substances (e.g. pathogens) are likely to be present. As another example, controller 37 may be operated to adjust the number of UV-LEDs 35 which are turned ON or OFF based on the flow speed of air through chamber 14 (e.g. a larger number of UV-LEDs 35 can be turned ON when a user breathes harder to ensure that air 5 flowing through chamber 14 receives sufficient dose of UV radiation 6). As another example, controller 37 may be operated to adjust the number of UV-LEDs 35 which are turned ON or OFF based on the humidity inside of chamber 14.

In some embodiments, controller 37 is operated to pulsate UV-LEDs 35 in a strobe like effect to limit the amount of time that they are turned on. Limiting the amount of time that a UV-LED 35 is turned on can help reduce the amount of heat given off by the UV-LED 35 and/or allow for temporary “over driving” of the UV-LED 35 to cause the UV-LED 35 to emit more UV in shorter bursts. In some embodiments, controller 37 is operated to control the voltage applied across each of the UV-LEDs 35 individually.

In some embodiments, controller 37 is operable to control the intensity of some or all of the UV-LEDs 35. Controller 37 may be operated to control the intensity of the one or more UV-LEDs 35 based on factors such as the physical location of device 10, the flow speed of air 5 through chamber 14, the humidity inside of chamber 14, etc.

In some embodiments, controller 37 is operated manually by a user 1 to control the UV-LEDs 35. In some embodiments, controller 37 is operated automatically (e.g. by an onboard computer 60 as described elsewhere herein) to control the UV-LEDs 35. In some embodiments, device 10 comprises both a manual mode of operation where controller 37 is operated manually and an automatic mode of operation where controller 37 is operated automatically. In these embodiments, a user 1 may switch between operating device 10 in its manual mode of operation and its automatic mode of operation.

Referring back to FIG. 1A, emitter 30 is located and/or oriented to direct suitable amounts of UV radiation 6 to air 5 as air 5 flows through chamber 14. For example, emitter may be provided at a location which is relatively proximate to the second longitudinal end 101B (i.e. at the longitudinal end of chamber 14 that is relatively proximate to outlet 18) and oriented to emit radiation 6 that is optically oriented towards first longitudinal end 101A (i.e. towards the longitudinal end of chamber 14 that is relatively proximate to inlet 16) as shown in FIG. 1A. Alternatively, emitter 30 may for example be provided at a location which is relatively proximate to the first longitudinal end 101A (i.e. at the longitudinal end of chamber 14 that is relatively proximate to inlet 16) and oriented to emit radiation 6 that is optically oriented in the direction towards second longitudinal end 101B (i.e. towards the longitudinal end of chamber 14 that is relatively proximate to outlet 18).

In embodiments where UV-LEDs 35 are arranged in an m×n array, the number of m UV-LEDs 35 may be spaced along a direction which is parallel to the second transverse axis 102B and the number of n UV-LEDs 35 may be spaced along a direction which is parallel to the first transverse axis 102A. In some embodiments, UV emitter 30 is oriented to align its principal optical axis with the axis 16A of inlet 16 and/or the axis 18A of outlet 18.

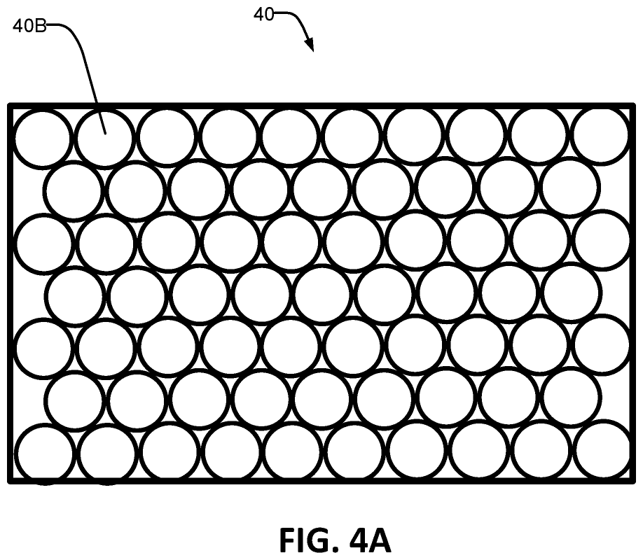

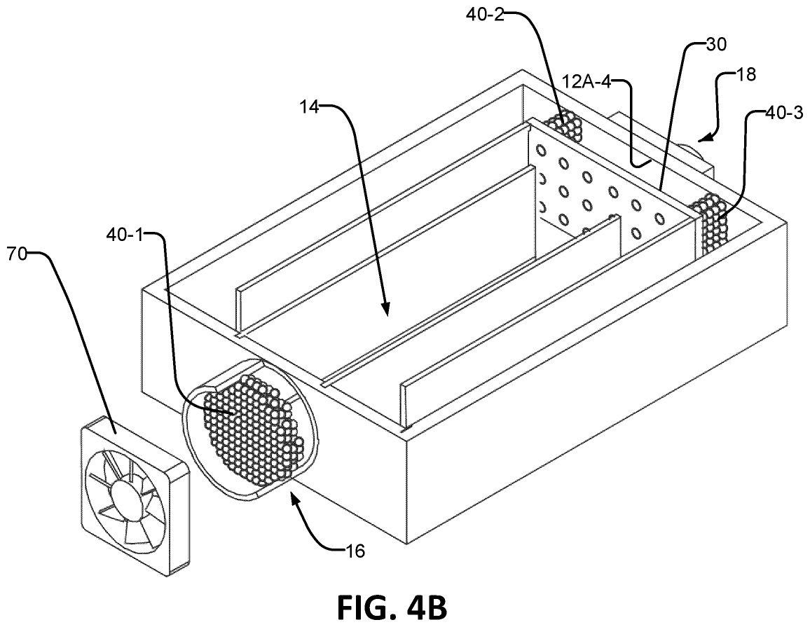

UV disinfection device 10 may optionally comprise one or more UV blockers 40 made of a suitable UV absorbing material (e.g. glass). UV blocker 40 is typically located at or relatively proximate to inlet 16 to prevent UV radiation 6 from escaping device 10. In the example embodiment shown in FIG. 1, UV blocker 40 is provided outside of chamber 14. In other embodiments, UV blocker 40 may be located inside of chamber 14 (e.g. see FIG. 4A).

FIG. 4 shows an exemplary embodiment of a UV blocker 40 provided outside of chamber 14. In the example embodiment shown in FIG. 4, UV blocker 40 is located outside of chamber 14. In some embodiments, UV blocker 40 comprises a UV reflective portion 40A sandwiched between two UV absorbing portions 40B. Reflective portion 40A comprises a reflective surface oriented to face toward inlet 16. Reflective portion 40A is provided to reflect UV radiation 6 that have escaped out of chamber 14. In some embodiments, the reflective surface of reflective portion 40A is aligned with the axis 16A of inlet 16. UV blocker 40 may also comprise UV absorbing portions 40B (i.e. portions made of tempered glass or materials of the like) located adjacent to reflective portion 40A. Advantageously, UV absorbing portions 40B can absorb residual UV radiation 6 (i.e. radiation that have escaped but are not reflected back into chamber 14), thereby preventing or otherwise reducing the amount of UV radiation 6 escaping from device 10 into the environment.

In some embodiments, UV emitter 30 emits small amounts of visible light in addition to UV radiation 6. In such embodiments, visible light can pass through UV absorbing portions 40B as UV radiation 6 is absorbed. In such embodiments, UV absorbing portions 40B can conveniently act as an indicator for whether UV emitter 30 is ON or OFF.

In some embodiments, UV blocker 40 comprises a bore 41 extending therethrough. Bore 41 may be located at reflective portion 40A as shown in FIG. 4. Bore 41 may be aligned with the principal optical axis of UV emitter 30 and/or the axis 16A of inlet 16. Bore 41 may be configured or shaped to receive a cap (not shown). In some embodiments, the cap comprises a reflective surface oriented to face toward inlet 16 (i.e. when the cap is coupled to bore 41). In other embodiments, the cap comprises sensors which are provided to detect UV light. In such embodiments, the cap may be coupled to UV blocker 40 (e.g. attached to UV block 40 to cover bore 41) to measure the intensity of UV radiation 6 at inlet 16 (i.e. to determine whether UV emitter 30 and/or UV LEDs 35 are operational or need replacement). In some embodiments, device 10 comprises a first cap having a reflective surface and a second cap having UV sensors, both of which may be removably coupled to UV blocker 40.

FIG. 4A depicts another exemplary embodiment of UV blocker 40. In the example embodiment illustrated in FIG. 4A, UV blocker 40 comprises a plurality glass beads 40B (e.g. glass spheres) pressed together in a packed configuration to prevent UV radiation from escaping device 10. In such embodiments, the space between adjacent glass beads 40B allows air to pass through UV blocker 40 while the glass beads 40B absorb the majority of radiation 6 escaping chamber 14. Depending on the orientation of UV emitter 30, UV blocker 40 may be provided at the inlet 16 and/or outlet 18 of UV housing 12. In the example embodiment illustrated in FIG. 4B, device 10 comprises a first UV blocker 40-1 located at inlet 16, and second and third UV blockers 40-2, 40-3 located between the rear surface of emitter 30 and the back interior surface 12A-4 of housing 12.

In other exemplary embodiments, UV blocker 40 comprises a slab of UV absorbing material 40B having pores spread throughout the slab to allow air to pass through UV blocker 40 while absorbing the majority of radiation 6 escaping chamber 14

Device 10 may optionally include one or more of the following additional systems and/or components:
one or more sensors 50 (e.g. VOC sensors, CO2 sensors, temperature sensors, pressure sensors, voltage sensors, current sensors, etc.) operable to measure or otherwise monitor air quality, temperature, humidity, and/or CO2 concentration inside chamber 14;
a low power computer 60 operable to gather, store, display and wirelessly transmit data collected by the one or more sensors 50;
a real time clock, which may be provided as part of computer 60, operable to track the running time of UV emitter 30;
a non-volatile memory, which may be provided as part of computer 60, operable to store data such as the elapsed running time of UV emitter 30;
one or more wireless communication devices (e.g. Bluetooth, wifi, NFC, etc.) which may be provided as part of computer 60;
an audible and/or visual light alarm, which may be provided as part of computer 60, operable to warn the user about device malfunction and/or various conditions such as low battery;
a global positioning system (GPS), which may be provided as part of computer 60, provided to track the location of device 10;
one or more fans 70 (e.g. air fans) operable to push or pull air through chamber 14 depending on, for example, whether the user 1 is inhaling air 5 or exhaling and/or depending on the CO2 levels inside chamber 14. Fans 70 may be provided inside of chamber 14 to receive radiation exposure (from UV emitter 30) which sterilizes fans 70. Sterilizing fans 70 advantageously allows the same device 10 to be used across multiple users by switching the direction of fan 70;
a display (e.g. an LCD) provided to show the various possible statuses of device 10;
a wireless charger operable to charge power source 32 without the need to physically remove power source 32 from device 10;
one or more electrostatic plates provided outside of inlet 16 to attract and/or trap charged dust particles in air 5 before air 5 flows into chamber 14;
a materials filter (e.g. a HEPA filter and/or a plain cloth filter) coupled to the inlet 16 and/or outlet 18 of device 10 to prevent unwanted substances from entering chamber 14;
one or more electronically controllable valves which can be operated to direct air flow 5 through device 10 to remove, for example, VOC and/or CO2 buildup from chamber 14;
a one way valve located at the inlet 16 and/or outlet 18 to regulate air flow in a single direction;
an audio jack output provided to allow the user 1 to plug in headphones or other audio broadcasting devices to hear the audible alarm;
one or more UV reflectors provided inside of chamber 14 and oriented to prevent or otherwise minimize the amount of UV light 6 escaping chamber 14 as shown in FIG. 1A; and
external light emitters (e.g. external LEDs emitting visible light) provided to show the status of device 10 and/or indicate alarm situations.

In embodiments where UV disinfection device 10 comprises an onboard computer 60, device 10 may be a smart device connected to the Internet of Things (IOT) to receive real-time updates from an external computer, a cloud server, etc. For example, device 10 may be connected to the IOT to receive notification that user 1 has entered a high risk area (e.g. a crowded area) and may alert the user 1 upon entering the high risk area. As another example, device 10 may be configured to automatically increase its UV output upon user 1 entering the high risk area.

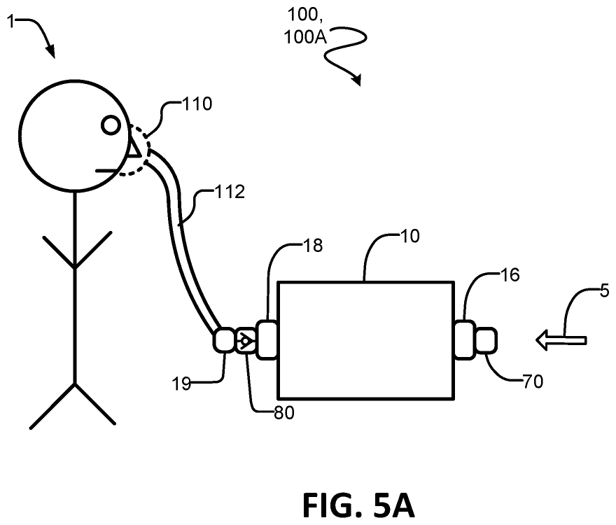

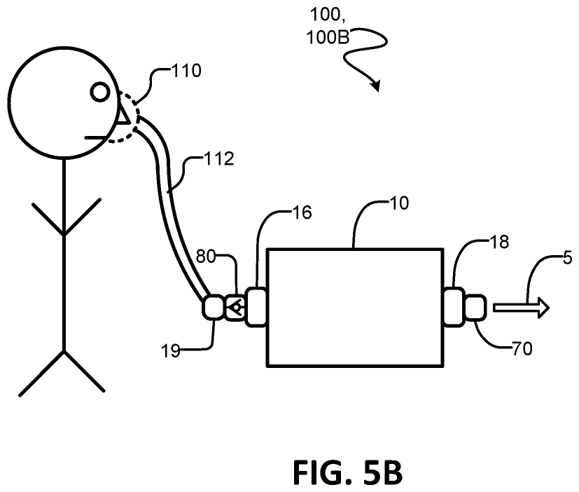

As described above, UV disinfection device 10 may be embodied as a part of a larger system. For example, UV disinfection device 10 may be embodied as a part of respiratory personal protective equipment (PPE). FIGS. 5A and 5B are schematic diagrams of a PPE 100 comprising a UV disinfection device 10 coupled to a face mask 110 through a conduit 112. Conduit 112 places UV disinfection device 10 in fluid communication with face mask 110. Device 10 may comprise an adapter 19 connecting inlet 16 or outlet 18 of device to conduit 112. Adaptor 19 may be configured to connect device 10 to different types of conduits 112 (e.g. different types of flexible holes, mask tubes, etc.).

Face mask 110 is provided to cover a mouth and at least a portion of a nose of a user 1. Face mask 110 may be a customized face mask or a commercially available face mask. Face mask 110 may be primarily made of filtration materials. For example, face mask 110 may be made of materials which have a pore size of less than about 0.3 microns to prevent user 1 from inhaling unwanted particles (e.g. pathogens, bacteria, viruses, etc.). Face mask 110 may comprise a suitable backing mechanism which is adjustable to secure face mask 110 snuggly against the face of user 1.

Referring now to FIG. 5A, an example embodiment of a respiratory personal protective equipment (PPE) 100A is shown. PPE 100A can be worn by a user 1 to neutralize unwanted particles (e.g. pathogens, bacteria, viruses, chemical contaminants, etc.) in air 5 as user 1 inhales air 5 through PPE 100A. For the purposes of facilitating the description, PPE 110A may be referred to herein as the “inhale embodiment” of PPE 100.

In some inhale embodiments, PPE 100A comprises a fan 70 configured to move air through device 10 and toward face mask 110. Fan 70 may be located at or near the inlet 16 of device 10 (e.g. see FIG. 4B) and configured to move air 5 from inlet 16 to outlet 18 as shown in FIG. 4. Alternatively, fan 70 may be located at or near the outlet 18 of device 10 and configured to draw air from the device 10 to user 1.

In some embodiments, PPE 100A comprises one way valve(s) configured to prevent air 5 that has been exposed to UV radiation from flowing back through device 10. For example, PPE 100A may comprise a one way valve 80 located at the outlet 18 of device 10 as shown in FIG. 4. Advantageously, one way valve 80 and/or fan 70 prevents user 1 from exhaling out of device 10.

Referring now to FIG. 5B, another example embodiment of a respiratory personal protective equipment (PPE) 100B is shown. PPE 100B is configured to neutralize unwanted particles (e.g. pathogens, bacteria, viruses, chemical contaminants, etc.) in air 5 as user 1 exhales through PPE 100B. For the purposes of facilitating the description, PPE 100B may be referred to herein as the “exhale embodiment” of PPE 100.

Like some inhale embodiments, some exhale embodiments 100B comprises a fan 70 and/or one way valves 80. In such embodiments, fan 70 is configured to move exhaled air 5 away from face mask 110 and through device 10. Fan 70 may be located at the outlet 18 of device 10 as shown in FIG. 5B or at the inlet 16 of device 10. In such embodiments, one way valve(s) 80 are configured to prevent air 5 that has been exposed to UV radiation 6 from flowing back toward user 1. For example, PPE 100B may comprise a one way valve 80 located at the inlet 16 of device 10 as shown in FIG. 5. Since UV radiation 6 can create ionized radicals which are not healthy to inhale, providing a one way valve 80 between user 1 and inlet 16 for the exhale embodiment can advantageously help reduce the chances of user 1 inhaling air 5 that has been treated with UV radiation 6.

In some embodiments, PPE 100A, 100B comprises a chemical materials filter (e.g. an activated charcoal filter). The materials filter may be located upstream of device 10 (i.e. located between user 1 and device 10) for the inhale embodiment 100A and downstream of device 10 (i.e. between device 10 and ambient environment) for the exhale embodiment 100B. For example, PPE 100A may comprise a materials filter located at the outlet 18 of device 10. Such filters are provided to capture volatile organic compounds or inorganic compounds that may be created or made more toxic after being exposed to the UV radiation as air 5 passes through the UV chamber 14.

In some embodiments, PPE 100A, 100B comprises an electrostatics filter (e.g. electrostatic plate(s)). The electrostatics filter may be located upstream of device 10 (i.e. located between user 1 and device 10) for the inhale embodiment 100A and downstream of device 10 (i.e. between device 10 and ambient environment) for the exhale embodiment 100B. For example, PPE 100A may comprise an electrostatics filter located at the outlet 18 of device 10. Such filters are provided to capture positive or negative ions that may be created after exposing air 5 passing through the UV chamber 14 to UV radiation 6.

In some embodiments, device 10 comprises one or more fluid flow sensors 50. Sensors 50 may be provided in chamber 14 to sense user 1 inhaling or exhaling through chamber 14. For example, in some inhale embodiments, sensors 50 may sense user 1 inhaling and activate fan 70 to move air through chamber 14 and toward user 1. As another example, in some exhale embodiments, sensors 50 may sense user 1 exhaling and activate fan 70 to move air through chamber 14 and away from user 1.

In some embodiments, face mask 110 comprises one or more vent holes (e.g. vents with small rubber flaps, small pores of the body of the face mask, etc.) that allow user 1 to inhale or exhale through the vent holes. In the inhale embodiments, fan 70 may be operated to direct a continuous stream of air 5 into mask 110 to thereby create a positive pressure environment inside face mask 110. This positive pressure environment helps prevent unwanted particles from entering the respiratory track of user 1 through the vent holes of face mask 110. In the exhale embodiments, fan 70 may be operated to direct air 5 away from mask 110 to thereby create a negative pressure environment inside face mask 110. This negative pressure environment encourages user 1 inhale air 5 through the vent holes of face mask 110 rather than through device 10.

In some embodiments, PPE 100 comprises a first device 14A provided to allow user 1 to inhale through first device 14A and a second device 14B provided to allow user 1 to exhale through second device 14B. In such embodiments, PPE 100 may comprise a first one-way valve coupled to the first device 14A to only allow air to flow towards face mask 110 and a second one-way valve coupled to the second device 14A to only allow air to flow away from face mask 110. Providing two devices 14 can help prevent exhaled air from mixing with inhaled air and other related carbon dioxide buildup issues.

A wide range of variations are possible within the scope of the present invention. These variations may be applied to all of the embodiments described above, as suited, and include, without limitation:
disinfection device 10 may comprise emitters which emit radiation of any suitable wavelength (e.g. UV emitter 30 may be replaced with emitter(s) which emit radiation having wavelengths outside of the UV spectrum);
disinfection device 10 may be used to deliver UV radiation (or other forms of radiation) to any fluid (e.g. disinfection device 10 is not limited to air purification applications);
UV emitter 30 may comprise any suitable type of UV radiation emitting devices (e.g. some or all of UV-LEDs 35 may be replaced with graphene LED devices or non-LED UV emitting devices).

Controller 37 or other controllers described herein may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, and the like. For example, one or more data processors in a control circuit for a device may implement methods as described (e.g. automatically controlling valves with a controller) herein by executing software instructions in a program memory accessible to the processors. It may be convenient to use a commercially available PLC for controller 37.

### Examples

Further aspects of the invention are further described with reference to the following examples, which are intended to be illustrative and not limiting in scope.

Simulations were conducted by the inventor using devices of the type described for device 10. The simulations were conducted to determine the reduction equivalent dose (RED) of a device (of the type as described for device 10) comprising interior surfaces made of aluminium which are specular reflective with a variable reflectance, UV-LEDs which are considered point sources each of which emitting 15 mW of power at 255 nm, and dividers made of quartz glass having 90% UV transmittance. RED refers to the UV dose derived by entering the log inactivation measured during a full-scale/virtual-scale reactor testing into a UV dose-response curve that was derived through collimated beam testing. RED values are specific to the challenge microorganism used during experimental testing and the validation test conditions for full-scale reactor testing. Simulations were conducted by the inventor under the assumption that air is non-absorbing, and that reflection and refraction are allowed at interfaces between air and quartz based on refractive indices.

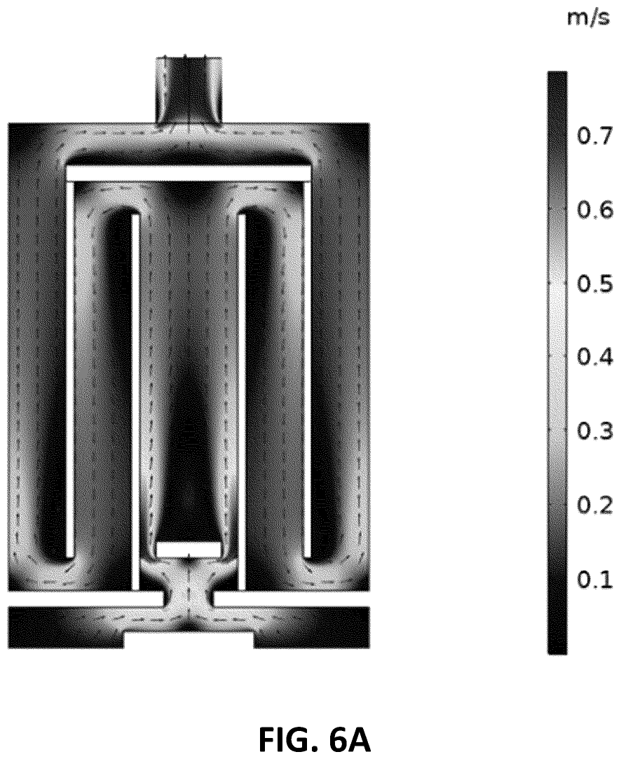

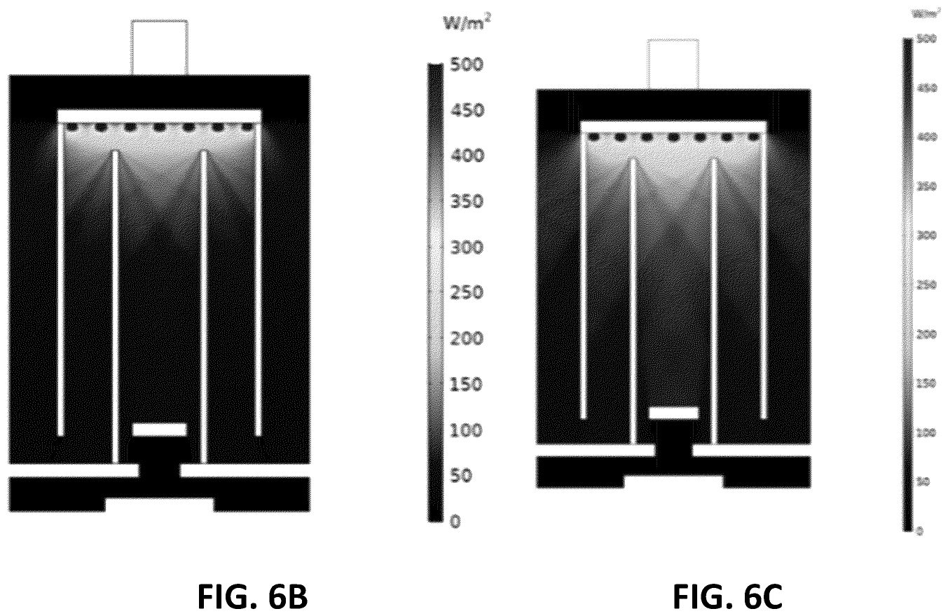

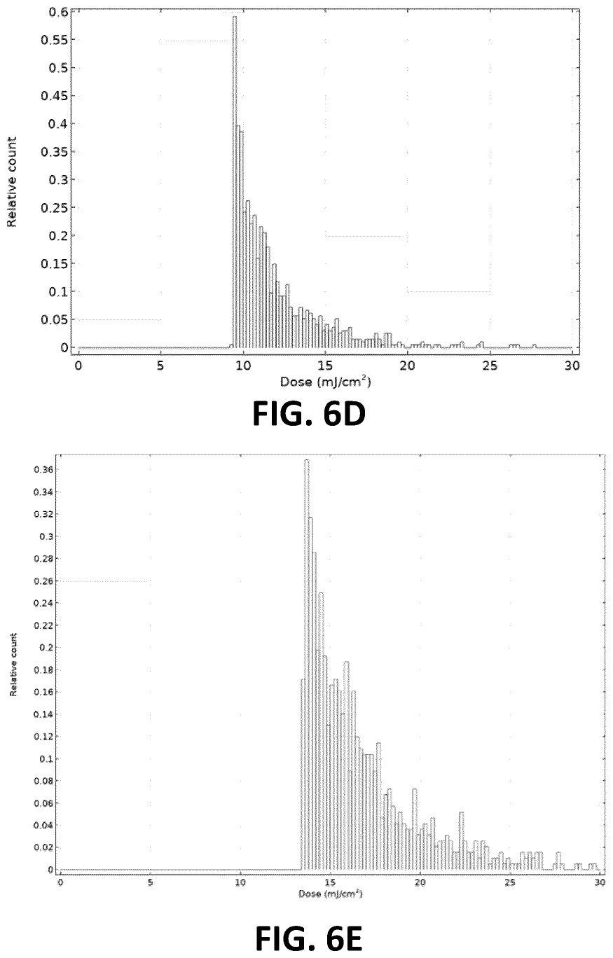

FIG. 6A depicts the simulated flow distribution of air flowing through the experimental device based on a RANS k-epsilon model. No-slip boundary conditions were applied to all walls (e.g. including dividers and the interior surfaces of the housing) except the inlet and the outlet. An inlet flow of 167 mL/s was applied on the two inlet surfaces (half on each) assuming a fully develop flow at these boundaries. The outlet boundary was assumed to be a zero pressure boundary. The air was assumed to be dry and its density and viscosity were calculated based on a temperature of 20° C.

FIG. 6B depicts the simulated fluence rate distribution of UV light inside of a chamber comprising interior surfaces made of aluminium which are specular reflective with 75% reflectance. FIG. 6C depicts the simulated fluence rate distribution of UV light inside of a chamber comprising interior surfaces made of aluminium which are specular reflective with 90% reflectance. The simulations were conducted based on a ray tracing model using an LED angular output pattern which allows for refraction and reflection at external and internal interfaces.

FIG. 6D depicts the simulated dose distribution of organisms travelling through a chamber comprising interior surfaces made of aluminium which are specular reflective with 75% reflectance. FIG. 6E depicts the simulated dose distribution of organisms travelling through a chamber comprising interior surfaces made of aluminium which are specular reflective with 90% reflectance. The dose accumulation of organisms travelling through the reactor is simulated using a particle tracing model. The particle tracing model uses the simulated flow field of the flow model (e.g. see FIG. 6A) and the simulated fluence rate distribution of the UV model (e.g. see FIGS. 6B and 6C) to calculate dose accumulation. The simulation was conducted by releasing 1,000 particles at the inlet. The particles have an initial velocity corresponding to the inlet velocity derived from the flow model. A bounce condition is used at all walls during the simulation.

The RED of a device was calculated based on the dose distribution. A log inactivation was calculated assuming a linear dose response curve (log I vs Dose) with 3-log inactivation at 7.5 mJ/cm2. The chamber comprising interior surfaces made of aluminium which are specular reflective with 75% reflectance had a log inactivation of 4.2 and a RED of 10.6 mJ/cm². The chamber comprising interior surfaces made of aluminium which are specular reflective with 90% reflectance had a log inactivation of 6.0 and a RED of 15.0 mJ/cm².

The examples and corresponding diagrams used herein are for illustrative purposes only. Different configurations and terminology can be used without departing from the principles expressed herein.

Although the invention has been described with reference to certain specific embodiments, various modifications thereof will be apparent to those skilled in the art without departing from the scope of the invention. The scope of the claims should not be limited by the illustrative embodiments set forth in the examples, but should be given the broadest interpretation consistent with the description as a whole. For example, various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An ultraviolet (UV) disinfection device comprising:
a housing shaped to define a chamber extending in a longitudinal direction;
an inlet located at a first longitudinal end of the chamber;
an outlet located at a second longitudinal end of the chamber;
a radiation emitter comprising one or more UV radiation sources, the radiation emitter located in the chamber between the inlet and the outlet, the radiation emitter spaced relatively distally from the first longitudinal end of the chamber and relatively proximately to the second longitudinal end of the chamber, the radiation emitter emitting UV radiation optically oriented toward the first longitudinal end of the chamber; and
a plurality of dividers located in the chamber, the plurality of dividers extending in the longitudinal direction and spaced apart in a transverse direction to define one or more flow paths between the inlet and the outlet,
wherein the plurality of dividers are made of a UV transparent material.

2. The UV disinfection device according to claim 1, wherein the plurality of dividers comprise first and second dividers extending from the radiation emitter toward the first longitudinal end.

3. The UV disinfection device according to claim 2, wherein the radiation emitter comprises first and second channels located at opposing edges of the radiation emitter, the first and second channels retaining a respective longitudinal end of the first and second dividers.

4. The UV disinfection device according to claim 3, wherein the plurality of dividers comprise third and fourth dividers extending from an interior surface of the housing at the first longitudinal end of the chamber toward the second longitudinal end.

5. The UV disinfection device according to claim 1, wherein the plurality of dividers are spaced apart to define first and second flow paths between the inlet and the outlet.

6. The UV disinfection device according to claim 5, wherein each of the first and second flow paths comprise respective segments that are serpentine shaped.

7. The UV disinfection device according to claim 6, wherein the serpentine shaped segments of the first and second flow paths are shaped to meander in opposing directions along the transverse direction.

8. The UV disinfection device according to claim 7, wherein the first flow path and the second flow path contain segments which overlap with each other.

9. The UV disinfection device according to claim 8, wherein the overlapping segments of the first and second flow paths are located in close proximity to the inlet.

10. The UV disinfection device according to claim 5, wherein the radiation emitter comprises a plurality of UV radiation sources, the plurality of UV radiation sources comprising a first UV-LED directing UV radiation optically oriented toward the first flow path and a second UV-LED directing UV radiation optically oriented toward the second flow path.

11. The UV disinfection device according to claim 1, wherein the inlet and the outlet comprise respective axes which are parallel to the longitudinal direction.

12. (canceled)

13. (canceled)

14. The UV disinfection device according to claim 11, wherein the radiation emitter comprises a principal optical axis which is parallel to the axes of the inlet and the outlet.

15. The UV disinfection device according to claim 1, wherein the one or more UV radiation sources comprise one or more UV light emitting diodes (UV-LEDs) arranged in a rectangular array.

16-19. (canceled)

20. The UV disinfection device according to claim 1, comprising a UV blocker located outside of the inlet, the UV blocker comprising a UV absorbing material.

21. The UV disinfection device according to claim 20, wherein the UV blocker comprises a reflective surface facing toward the inlet, the reflective surface located between a first portion of the UV absorbing material and a second portion of the UV absorbing material.

22. The UV disinfection device according to claim 21, wherein the reflective surface is aligned with the axis of the inlet.

23. The UV disinfection device according to claim 1, comprising one or more UV blockers made of glass beads pressed together in a packed configuration.

24. The UV disinfection device according to claim 23, comprising a first UV blocker located at the inlet.

25. The UV disinfection device according to claim 24, comprising second and third UV blockers located between a rear interior surface of the housing and a rear surface of the radiation emitter.

26. A personal protective equipment comprising:
a face mask;
the UV disinfection device according to claim 1;
a one-way valve located between the disinfection chamber and the face mask, the one-way valve configured to prevent air from back flowing from the disinfection chamber toward the face mask; and
a fan configured to move exhaled air from the face mask through the UV disinfection chamber to ambient environment.

27-34. (canceled)
